(19) United States
(12) Patent Application Publication
(10) Pub. No.: US 2025/0320450 A1
(43) Pub. Date: Oct. 16, 2025

(54) METHOD OF ENCAPSULATION BY ELECTROHYDRODYNAMICS

(71) Applicant: Chr. Hansen A/S, Hoersholm (DK)

(72) Inventors: Panagiota DIMA, Kgs. Lyngby (DK); Ana Carina Loureiro MENDES, Kgs. Lyngby (DK); Peter Reimer STUBBE, Kgs. Lyngby (DK); loannis S. CHRONAKIS, Kgs. Lyngby (DK); Surender Kumar DHAYAL, Hoersholm (DK)

(73) Assignee: Chr. Hansen A/S, Hoersholm (DK)

(21) Appl. No.: 18/873,509

(22) PCT Filed: Jun. 15, 2023
(86) PCT No.: PCT/EP2023/066136

§ 371 Date: Dec. 10, 2024

(30) Foreign Application Priority Data
Jun. 16, 2022 (EP) 22179401.9

(51) Int. Cl. C12M 1/00; B01D 1/14; B01D 1/18; B05B 5/16; C12N 1/20; F26B 3/12

(52) U.S. Cl. CPC C12M 47/14; B01D 1/14; B01D 1/18; B05B 5/1683; C12N 1/205; F26B 3/12

(57) ABSTRACT

The present disclosure relates to a process for electrostatic spray drying of a living microorganism with a specific surface charge. using electrostatic charge provided by a high voltage source, wherein said electrostatic charge is the same as the surface charge of the microorganism.

### FIELD OF THE INVENTION

The present disclosure relates to methods for electrostatic spray drying of microorganisms and to microorganisms embedded in dried particles.

### BACKGROUND OF THE INVENTION

Spray drying is a technique wherein a suspension is atomized (or sprayed) and thereafter rapidly dried by the use of a gaseous hot drying medium. The scalability of the manufacturing process allows for the formation of particles, destined for use in a wide number of industries, including food, polymer, biotechnology, pharmaceutical or and medical. The choice of atomizer for breaking up the feedstock (suspension) into droplets depends to a large extent on the type of solution and the desired characteristics of the dried particles. Conventional atomizers include rotary atomizers, relying on the use of centrifugal force for droplet formation, hydraulic nozzle atomizers, relying on pressure, and pneumatic nozzle atomizers, relying on kinetic energy. Spray drying has been applied to a wide number of particle types, including bacteria and other microorganisms.

Electrospraying is an electohydrodynamic method used for encapsulation of compounds or bacterial cells in a polymer matrix. Electrostatic droplet formation has recently been disclosed as an improved way to achieve droplet formation. The method relies on electrostatically charging the suspension prior to droplet formation. The charging of the suspension may result in improved drying characteristics of the formed droplets and may facilitate particle collection and aggregation. Although electrostatic spray drying offers a significant number of advantages over conventional spray drying, the use and its application to drying of microorganisms has not yet been successful. This is logic as it is known that the viability of microorganisms is affected by electrostatic charges, in fact high voltage is a common method for disinfection of liquids. Consequently, the use of electrostatic spray drying has mostly been focused on drying of non-living matter.

The patent publication WO2021152111A1 discloses a method for electrostatic spray drying of a living microorganism. However, there still is a need for providing improved methods for electrostatic spray drying, and particularly methods leading to cost savings and/or improved stability of the final product.

### SUMMARY OF THE INVENTION

It has been observed that electrical fields and charge polarities during spray drying, can be optimized to improved encapsulation/immobilization of bioactive compounds, such as microorganisms, by affecting nano-microstructures using electohydrodynamics.

The present invention provides an improved method for electrostatic spray drying of a living microorganism, by utilizing a high voltage source that is negatively charged to encapsulate a microorganism with a negative surface charge.

The microorganism may inherently have a negative surface charge, or the microorganism may be treated such as to acquire a negative surface charge.

According to a first aspect of the invention, a process for electrostatic spray drying of a living microorganism is provided, said process comprising the following steps: a) providing a suspension, comprising a microorganism with an electrical surface charge and a formulation aid; b) applying an electrostatic charge to said suspension; c) forming droplets of said suspension; d) drying said droplets, thereby forming dried particles; and e) collecting the dried particles; wherein the electrostatic charge has the same polarity as the electrical surface charge of the microorganism.

In one embodiment, the electrostatic charged is applied to the suspension, while simultaneously moving the suspension.

In one embodiment, the collecting of dried particles is achieved using a collector with the opposite charge compared to the electrostatic charge and the electrical surface charge of the microorganism.

In one embodiment, the microorganism with a negative surface charge is selected from the group consisting of Bifidobacterium animalis, Lactobacillus acidophilus, Pseudomonas aeruginosa and Escherichia coli.

In one embodiment, the microorganism is Bifidobacterium animalis subsp. lactis (DSM 15954).

In one embodiment, the shape of dried particles is a sphere, fiber, spiral, rod or hybrid.

In one embodiment, the formulation aid is a polysaccharide, protein, lipid or synthetic polymer, or a mix thereof.

In one embodiment, the polysaccharides are either positively charged, negatively charged or neutral.

In one embodiment, the polysaccharides are selected from the group consisting of maltodextrin, starch, xanthan, gellan, alginate, pectin, glucan and chitosan.

In one embodiment, the proteins are dairy proteins, non-dairy animal proteins, plant proteins, algae proteins or fermentation product proteins.

According to a second aspect of the invention, use of a positive-charge or negative-charge high voltage source to encapsulate a living microorganism is provided, said microorganism having a surface charge, which is the same as the charge of the high voltage source.

According to a third aspect of the invention, a particle comprising a polymer and at least one cell of a living microorganism is provided, wherein the at least one cell is located in a core of the particle, and wherein the polymer composition of the particle is uniform and forms a shell around the core that is substantially devoid of cell(s).

According to a fourth aspect of the invention, a particle comprising a living microorganism is provided, obtainable by a process according to the first aspect.

According to a fifth aspect, use of a particle according the third or fourth aspect, to manufacture a food product, a feed product, a dietary supplement or a pharmaceutical product is provided.

According to a sixth aspect, a food product, a feed product, a dietary supplement or a pharmaceutical product, is provided comprising a particle according the third and fourth aspect.

### BRIEF DESCRIPTION OF THE FIGURES

FIG. 1 is a schematic representation of the EHD drying setup.

FIG. 2 are two graphs showing effect of polarity on water loss (A) and viability (B) of EHD dried BIFIDO probiotics. Each pair of samples (S1-S2, S3-S4, S5-S6) present statistically significant differences (p<0.05) between results and the reference sample (R0) is denoted with a different letter.

FIG. 3 are two graphs showing effect of distance between needles and the sample holder on water loss (A) and viability (B) of EHD dried BIFIDO probiotics. Each pair of samples (S1-S7, S5-S9) that present statistically significant differences (p<0.05) between results and the reference sample (R0) is denoted with a different letter.

FIG. 4 are two graphs showing effect of temperature on water loss (A) and viability (B) of EHD dried BIFIDO probiotics. The samples (S11, S14, S16) that present statistically significant differences (p<0.05) between results and their corresponding reference sample (R0, R13, R15) are denoted with a different letter.

FIG. 5 are two graphs showing effect of needles number and distance between the needles and the moving filter, on water loss (A) and viability (B) of EHD dried BIFIDO probiotics. The samples (MF4, MF5, MF2, MF3) that present statistically significant differences (p<0.05) between results and the reference sample (R0)) are denoted with a different letter.

FIG. 6 are two graphs showing effect of needle number and distance between the needles and the moving filter, on water loss (A) and viability (B) of EHD dried BIFIDO probiotics. The samples (MF4, MF5, MF2, MF3) that present statistically significant differences (p<0.05) between results and the reference sample (R0)) are denoted with a different letter.

FIG. 7 are two graphs showing effect of different drying setups on water loss (A) and viability (B) of BIFIDO probiotics dried for 2 hours. The samples (S1, SM1, MF4, FD, FDM) that present statistically significant differences (p<0.05) between results and the reference samples (R0, R17) are denoted with a different letter.

FIG. 8 is a graph showing FTIR spectra of BIFIDO probiotic bacteria dried for 2 hours. The samples S1, MF4 and FD were dried in different setups and the reference sample R0 was non-dried BIFIDO.

FIG. 9 is a graph showing FTIR spectra of BIFIDO probiotic bacteria dried for 2 hours with and without the addition of maltodextrin. The samples S1, SM1 and FD, FDM were dried in different setups.

FIG. 10 are two graphs showing effect of different drying setups on zeta potential (A) and hydrophobicity (B) of BIFIDO probiotics dried for 2 hours. The samples (S1, SM1, MF4, FD, FDM) that present statistically significant differences (p<0.05) between results and the reference samples (R0, R17) are denoted with a different letter.

FIG. 11 is a graph showing zeta potential of probiotic bacteria in KH₂PO₄ at different pH values.

FIG. 12 is a graph showing cell surface Hydrophobicity of probiotic cells at different pH (expressed as % of adhesion to hexadecane.

FIG. 13 are confocal laser scanning microscopy micrographs showing effect of voltage and polarity on the cell ‘self-organization’ within the microcapsules.

FIG. 14 Confocal laser scanning microscopy micrographs showing cell distribution within the electrosprayed microcapsules.

FIG. 15 are two graphs showing Effect electric field polarity and voltage on the glass transition temperature (Tg) of electrosprayed microcapsules. FIG. 15A shows data for cells in 75% w/v maltodextrin and 10% v/v, and FIG. 15B shows data for cells in 75% w/v maltodextrin.

FIG. 16 is a FTIR spectra of probiotic bacteria with and without the influence of an external electric field.

FIG. 17 is a graph showing viability loss of encapsulated cells electrosprayed using negative and positive polarity and non-encapsulated (free cells).

### DETAILED DESCRIPTION OF THE INVENTION

The inventors have found that by utilizing electrohydrodynamics, encapsulation of microorganisms can be achieved resulting in cost savings and/or improved process efficiency and/or improved stability of the final product.

This is achieved through a process for electrostatic spray drying of a living microorganism, the process comprising the steps of providing a suspension, comprising a microorganism with an electrical surface charge and a formulation aid; applying an electrostatic charge to said suspension; forming droplets of said suspension; drying said droplets, thereby forming dried particles; and collecting the dried particles. The electrostatic charge should be the same polarity as the electrical surface charge of the microorganism. This results in a particle comprising a polymer and at least one cell of a living microorganism, wherein the at least one cell is located in a core of the particle, and wherein the polymer composition of the particle is uniform and forms a shell around the core that is substantially devoid of cell(s). Such particle structure protects the microorganisms, thereby increasing the stability of the product.

It is also possible to utilize electrohydrodynamics to achieve particles with microorganisms close to the surface, rather than in the core of the particle. This is achieved by applying an electrostatic charge which is the opposite of the surface charge of the microorganism.

Preferably the suspension comprises one or more formulation aids. At least part of the formulation aids may be added to the suspension prior to the application of an electrostatic charge to the suspension. The formulation aids are typically provided as a drying protectant wherein said drying protectant acts to stabilize the microorganism within the suspension, the droplets, and/or the dried particles. Preferably, the drying protectant is selected such that is act to decrease, such as to prevent, killing of said microorganisms, both during the electrostatic spray drying process itself, but also during subsequent use of said dried particles and/or the microorganisms, including storage, transport and/or additional processing.

The present invention further relates to a particle comprising living microorganisms embedded in a mass of formulation aids. The particle is preferably compact, such that the particle has a total inner void volume below 5% of the total volume of the particle. Furthermore, in a preferred embodiment of the present disclosure, the microorganisms are not present at the surface of the particle. Consequently, the microorganisms are preferably embedded within the formulation aid of the dried particles. In an embodiment of the present disclosure, the dried particle comprises substantially a single phase, in addition to the microorganisms. Preferably, the dried particles are thereby not a layered dried particle, such as a dried homogeneous particle encapsulated in a protective layer. In a typical embodiment of the present disclosure, the dried particle has a substantially continuous radial gradient of formulation aids. The dried particles may thereby have a high concentration of formulation aids at the surface, such as 100%. The concentration of formulation aids may continuously decrease towards the center of the particles, thereby forming a radial gradient of formulation aids. Preferably the center of the particles has the highest concentration of microorganisms.

In an embodiment of the present disclosure the electrostatic charge has been applied such that polar components are forced towards the surface of the droplets, while less polar components of the suspension are forced towards the center of the droplets. Typically the polarity of the different components have an impact on the resulting electrostatic charge distribution of the formed droplets. For example, electrons provided to the suspension are typically associated with the more polar components, usually the solvent, resulting in an electrostatic repulsion between the polar components forcing the polar solvent and polar formulation aids dissolved in the solvent towards the surface of the droplets. The similar effect may furthermore act to force the microorganisms towards the center of the droplets, encapsulated by the formulation aid. Forcing the solvent towards the surface typically results in faster evaporation rates, while embedding the microorganisms in the center of the droplets results in improved encapsulation of the microorganisms.

In an embodiment of the present disclosure the electrostatic charge is applied to the suspension by an electrode in contact with said suspension, and wherein the electrode has a pulsed electric potential difference, with respect to ground. Preferably the electric potential difference, the voltage, has a constant polarity. The electrode may thereby be part of a direct current (DC) circuit wherein current flows in one direction only and the electrode is always kept negative or positive. The formed droplets may thereby all have an overall negative charge or an overall positive charge. Typically, pulsation of the electric potential difference leads to improved electrical charging of the suspension, and furthermore it may lead to improved characteristics of the dried particles.

In an embodiment of the present disclosure the electrostatic charge is applied to the suspension by contacting said suspension with at least one electrode having an electric potential difference with respect to ground, a voltage. The electrode may therefore be provided in a configuration for applying said voltage to said suspension. Additional electrodes may be provided, such as two, or three, or four, or even additional electrodes for applying said electrostatic charge to said suspension. Preferably the two or more electrodes have the same polarity, such as positive or negative voltage, as given for example in a direct current circuit. Configurations of electrodes for applying an electrostatic charge are known by a person skilled in the art, and may include the use of specific materials, surface area, and shape of the one or more electrodes.

In an embodiment of the present disclosure the electrode has an electric potential difference, with respect to ground, below about 40 kV, such as below about 35 kV, such as below about 30 kV, such as below about 25 kV, such as below about 20 kV, such as below about 15 kV, such as below about 10 kV. Preferably the voltage is sufficiently low in order to not cause any damage to the microorganisms. Thereby, the voltage should be sufficiently low in order to not kill the living microorganisms.

In an embodiment of the present disclosure the electrode has a fixed polarity, with respect to ground, such as fixed negative polarity or fixed positive polarity. The electrode, or the multiple electrodes, may therefore be configured for applying a direct circuit (DC) voltage. Preferably, the electrode(s) is configured for a continuous supply of electrons, or a continuous drain of electrons, to/from the suspension. Typically, a positive electrode, an anode, drains a suspension of electrons while a negative electrode, a cathode, supplies a suspension with electrons. The electrode may temporarily have a ground potential, i.e. 0 V.

In an embodiment of the present disclosure the electric potential difference, with respect to ground, of the electrode is constant. The voltage of the electrode may thereby be constant, and consequently, the electrostatic charge may be delivered to the suspension by the use of a constant voltage.

In another embodiment of the present disclosure the electric potential difference, with respect to ground, of the electrode varies over time, such as in periodic variations. The periodic variations may be described by a wave function, such as by a sinus wave, or a combination of multiple wave functions, combined to make a periodic variation. The periodic variations may be described by two or more voltage levels, which the voltage of the electrode varies, in cyclic variations. One of the voltage levels may be ground.

In an embodiment of the present disclosure the electric potential difference, with respect to ground, of the electrode varies periodically, such as in a periodic step function. The voltage of the electrode may vary according to any function and may furthermore depend on parameters of the method, such as the feed rate of the suspension, the droplet sizes, the contents of suspension, such as the type of components and their relative ratios, and furthermore desired parameters of the dried particles.

In an embodiment of the present disclosure the electric potential of the electrode is applied by pulse width modulation, such as by a square wave. The voltage of the electrode may as a consequence vary between two or more set levels, forming a square wave, wherein the time between two pulses may be a set value or may vary depending on parameters of the processing method as mentioned elsewhere herein. The voltage may be provided as a pulse between two or more voltage values, wherein the dwell time at each level may be set individually, and wherein one of the voltage levels may be 0 V.

In an embodiment of the present disclosure the components of the suspension are partitioned within the formed droplets with respect to their polarity, such as for increased evaporation of the solvent and/or increased encapsulation of the microorganism. Partitioning of components of the suspension may lead to advantageous properties of the formed droplets, such as increased evaporation, e.g. decrease evaporation time and/or less water content in the final dried particles, and/or improved encapsulation of the dried particles. Consequently, components of the suspension may be chosen based on how they are partitioned within a formed droplet, such as in an electrostatically charged formed droplet.

In an embodiment of the present disclosure the components of the suspension of higher polarity are partitioned to the surface of the droplets and components of the suspension of lower polarity are partitioned to the center of the droplets. In an embodiment of the present disclosure at least two components of said suspension have different dielectric properties. Preferably, there is a relationship between the partitioning of the components of the suspension, within the droplets, and the dielectric properties and/or the effective dielectric properties, of said components of the suspension.

In an embodiment of the present disclosure the microorganism has a lower effective dielectric property than the formulation aid and/or the solvent. In an embodiment of the present disclosure the solvent has a higher dielectric constant than the formulation aid, and the formulation aid has a higher dielectric constant than the microorganism. Among the solvent, the formulation aids and the microorganism, it is preferred that the solvent has the highest dielectric property, while the formulation aid has a higher dielectric property than the microorganisms. The dielectric property of the microorganisms may be measured and/or given as an effective dielectric property, wherein the overall dielectric property of the microorganism is given, and not the dielectric property of individual components, such as specific membrane proteins, as known by a person skilled in the art, for example in Sanchis et al., Dielectric characterization of bacterial cells using dielectrophoresis, Bioelectromagnetics, 2007.

In an embodiment of the present disclosure the droplets are formed by atomizing the suspension. Atomizing and spraying may be used interchangeably herein as referring to the process of forming multiple small droplets of a liquid, such as a suspension, from a larger volume, such as a feedstock. Droplet formation, i.e. the breakup of a liquid volume into smaller droplets requires energy, due to the increase in surface area. The interfacial energy, typically given at the liquid-air interface at the surface of the droplets, requires the addition of energy for formation. As known to a person skilled in the art, the energy may be supplied in a wide range of ways.

In an embodiment of the present disclosure the formation of droplets is carried out by means of an atomizing device, such as an ultrasound nozzle; a pressure nozzle; a two-fluid nozzle (e.g. using CO₂ or N₂ or other gases as atomizing gas); a vibrating nozzle; a frequency nozzle, an electrostatic nozzle; or a rotating atomizing device. Different types of nozzles are known to a person skilled in the art, and they may have their individual advantages. Several parameters can be adjusted based on the desired properties of the droplets, and consequently the dried particles, including the flow rates of an atomizing gas, the flow rate of the suspension/feedstock, the use of surfactants, the configuration of the nozzle, the type of nozzle, the forces acting on the suspension (gravity, electrical, centrifugal or other).

In an embodiment of the present disclosure the formation of droplets is carried out by means of a two-fluid nozzle. Two-fluid nozzles atomize a liquid, such as a suspension, by an interaction between a high velocity gas and a liquid, such as a suspension.

Typically compressed air is used as an atomizing gas, but other gases, such as steam may be used. A two-fluid nozzle may be of an internal mix type or an external mix type depending on the mixing point of the gas and liquid streams relative to the nozzle face.

In an embodiment of the present disclosure the formation of droplets in step c) is performed using an atomizing gas. Preferably the atomization of the droplets is performed by the use of a two-fluid nozzle configured for droplet formation by the use of said atomizing gas.

In an embodiment of the present disclosure the atomizing gas is selected from the group consisting of an inert gas (such as Nitrogen and Carbon dioxide), a noble gas (e.g. Helium, Argon or Neon), and an alkane gas (such methane), or a mixture thereof.

In an embodiment of the present disclosure the atomizing gas comprises or consists of Nitrogen, Carbon Dioxide and/or atmospheric gas, or a mixture thereof. The gas may be treated before use in any suitable way including filtered, sterilized, and/or dehumidified. However, in an embodiment of the present disclosure the atomizing gas has not been dehumidified. The use of non-dehumidified gas may be advantageous in several aspects, including providing a simpler operation, decreasing cost and time, and may furthermore lead to better droplet and/or dried particle characteristics.

In an embodiment of the present disclosure the atomizing gas has a moisture content below about 1000 ppm, such as below about 500 ppm, such as about below 100 ppm, such as about below 50 ppm, such as about below 10 ppm.

In an embodiment of the present disclosure the droplet forming step, (e.g. the spray step) is carried out with an atomizing gas inlet temperature of at most about 200° C., such as in the range between about 20° C. to about 200° C., such as in the range between about 40° C. to about 150° C., or such as in the range between about 40° C. to about 120° C., such as between about 40° C. to about 90° C., such as between about 50° C. to about 90° C., such as between about 60° C. to about 85° C., such as about 80° C. It may be desirable to have an elevated temperature, with respect to normal room temperature for optimal droplet formation, solvent evaporation and/or dried particle characteristics. However, an increased temperature may at the same time be detrimental for components of the suspension, such as the microorganisms. Therefore, the optimal temperature may not only be based on easiest mode of performing said process, such as at room temperature, but instead may be a contribution of multiple factors, including ease of performing said process, rapid evaporation, viability of the living microorganisms, specific characteristics of the dried particles, such as optical encapsulation of the microorganisms, a sufficiently low volume of gas-filled voids within the dried particles (compactness of the dried particles) and other parameters. Consequently, the optimal temperature is non-trivial and is based on several factors. Preferably the temperature is above room temperature, such as for rapid evaporation, but below a temperature detrimental to the microorganisms, given the specific temperature experienced by said microorganisms, as it is preferably thermally isolated during drying, by being embedded within the formulation aid and, at least temporarily, the solvent. The temperature may consequently, be within the range of between about 20° C. to about 200° C.

In an embodiment of the present disclosure the atomizing gas has an inlet pressure in the range between about 1 kPa to about 500 kPa, such as in the range between about 5 kPa to about 500 kPa, such as in the range between about 5 kPa to about 300 kPa, such as in the range between about 5 kPa to about 100 kPa, such as about 60 kPa, or such as about 70 kPa, or such as about 80 kPa, or such as in the range between about 100 kPa to about 400 kPa, such as about 120 kPa, or about 150 kPa, or about 200 kPa, or about 250 kPa, or about 300 kPa, or about 350 kPa.

In an embodiment of the present disclosure the atomizing gas has an inlet pressure in the range between about 50 kPa to about 400 kPa. Typically, the inlet pressure of the atomizing gas is defined as the pressure of said atomizing gas before being supplied to the nozzle, such as the two-fluid nozzle. Typically, the inlet pressure of the atomizing gas is the pressure of said atomizing gas supplied to a spray drying apparatus, such as an electrostatic spray drying apparatus. The gas may be supplied from a gas tank comprising at least one pressure regulator, for controlling the pressure provided to said spray dryer and/or said nozzle. Preferably, the pressure supplied to the nozzle is substantially the same as the inlet pressure. Following formation of droplets, the pressure of the atomizing gas decreases, typically due to high resistance in the atomizing nozzle in combination with a large cross section of the drying chamber.

In an embodiment of the present disclosure the dried particles have a size from about 1 micrometer to about 800 micrometers, such as in the range from about 5 micrometers to about 800 micrometers, such as about 10 micrometers to about 600 micrometers, such as about 10 micrometers to about 300 micrometers, such as about 10 micrometers to about 200 micrometers, such as about 10 micrometers to about 50 micrometers, or such as about 50 micrometers to about 200 micrometers, such as about 50 micrometers to about 100 micrometers, such as about 75 micrometers, or such as about 100 micrometers to about 200 micrometers, such as about 150 micrometers, measured as Dv50 values. As known to a person skilled in the art Dv50 is typically used for referring to the median of a volume distribution.

In an embodiment of the present disclosure the size distribution of the dried particles is substantially unimodal.

In an embodiment of the present disclosure the dried particles are substantially dry. Small amounts of solvent, such as trace amounts of solvent, may be present in the dried particles. Therefore in an embodiment of the present disclosure the process comprising a drying step for drying the formed droplets.

In an embodiment of the present disclosure the process comprising a drying step, and wherein the drying of the droplets takes place under reduced pressure. A reduced pressure may be used in order to improve the evaporation, such as to increase the evaporation rate, and furthermore to control or steer the dried particles into a dedicated collector at an outlet end of the drying chamber, wherein the collector may be in the form of a filter or a sieve. A reduced pressure may have additional advantages, such as for recirculation of the atomizing gas.

In an embodiment of the present disclosure the process comprises a drying step of the formed droplets (wet particles), and wherein the water activity (a_{w}) of the dried particles is below about 1.0, such as in the range of about 0.01 to about 0.6, such as about 0.05 to about 0.5, such as about 0.1 to about 0.5, such as about 0.2, or such as about 0.3, or such as about 0.4. Water activity (a_{w}) is the partial vapor pressure of water in a substance divided by the standard state partial vapor pressure of water. Wherein the standard state is defined as the partial vapor pressure of pure water at the same temperature. In one embodiment of the present disclosure the water activity is chosen based on the type microorganisms, such as wherein the level of water activity is set to a value wherein said type of microorganisms is advantageously dried, such as wherein the viability is not significantly affected.

In an embodiment of the present disclosure the process comprises a drying step of the formed droplets (wet particles), and wherein the solvent (e.g. water) content of the dried particles is below about 20% by weight, such as below about 15% by weight, such as below about 10% by weight, such as below about 5% by weight, such as below about 3% by weight, such as below about 1% by weight, such as below about 0.1% by weight, with respect to the total weight of the dried particles.

In an embodiment of the present disclosure the solvent (e.g. water) content of the dried particles is below about 10% by weight, (preferably below about 5%, or below about 1% by weight), with respect to the total weight of the dried particles. Small amounts of water may consequently be present following drying of the formed droplets, and may even be advantageously, in that it may be beneficial for increased viability of the microorganisms.

In an embodiment of the present disclosure the dried particles are collected at an outlet end of a drying chamber. The outlet end may further be equipped with a collector for collection of the dried particles, such as a filter, a container, and/or a sieve.

In an embodiment of the present disclosure the dried particles are collected at the outlet end of the drying chamber using a filter (such as an electrostatic filter) or a sieve. Steric interactions preferably obstructs the passage of particles, while gas, such as the atomizing gas, is allowed to pass through the collector.

In an embodiment of the present disclosure the dried particles are collected at the outlet end of the drying chamber using a sieve having an aperture diameter below about 500 micrometers, such as in the range between about 40 micrometers to about 300 micrometers, such as in the range from about 50 micrometers to about 250 micrometers, such as about 50 micrometers, such as about 100 micrometers, such as about 150 micrometers, such as about 200 micrometers or such as about 250 micrometer. Preferably the aperture diameter is below the particle size, given by Dv50, more preferably below Dv30, even more preferably below Dv10, yet even more preferably below Dv5, such as below Dv3, such as below Dv1. The apertures may be provided in a suitable shape, such as round openings and/or slits (grating). In general, the smallest dimension of an opening is the relevant size for obstruction of particles. Thereby, for slits, the aperture diameter refers to the slit width, the same reasoning applies to other shapes of openings.

In an embodiment of the present disclosure the dried particles are collected at the outlet end of the drying chamber using a sieve having an aperture diameter in the range from about 40 micrometers to about 300 micrometer.

### Deposit and Expert Solution

The applicant requests that a sample of the deposited microorganisms stated below may only be made available to an expert, subject to available provisions governed by Industrial Property Offices of States Party to the Budapest Treaty, until the date on which the patent is granted.

Deposits were made at a Depositary institution having acquired the status of international depositary authority under the Budapest Treaty on the International Recognition of the Deposit of Microorganisms for the Purposes of Patent Procedure: Leibniz Institute DSMZ-German Collection of Microorganisms and Cell Cultures Inhoffenstr. 7B, 38124 Braunschweig, Germany. Accession number and deposit details are shown in Table 1.

TABLE 1
Deposit information

| Strain | Accession No. | Deposit date |
|---|---|---|
| Bifidobacterium animalis subsp. lactis | DSM 15954 | 2003 Sept. 30 |

### EXAMPLES

### Example 1

### Materials and Methods

Maltodextrin (Dextrose Equivalent 12), commercial name Glucidex IT12, was obtained from Roquette (Lestrem, France). Sodium chloride (NaCl) (Merck, Kenilworth, NJ, USA) and tryptone (Oxoid, Hampshire, UK) isotonic solutions were prepared for the bacterial dilutions. The bacterial viability measurements were executed using the dual staining LIVE/DEAD™ BacLight™ Bacterial Viability Kit (L-7012, Molecular Probes, Eugene, OR, USA). The probiotic cells Bifidobacterium animalis, DSM 15954 (in the following called BIFIDO) of concentration 3×10¹¹ cells/gram were provided by Chr. Hansen A/S (Hørsholm, Denmark) and stored −80° C. until further use.

### Electrohydrodynamic Drying

The probiotics (3×10¹¹ cells/gram) stored at −80° C., were thawed at room temperature and transferred (5 g) to an aluminum sample holder 55 mm×80 mm. The samples were placed inside a closed polycarbonate chamber (FIG. 1), where temperature and relative humidity (RH=20±3%) were controlled by introducing air or nitrogen gas through a diffuser.

The EHD drying setup consisted of a unipolar DC high voltage generator (Gamma High Voltage Research, Florida, USA) coupled with a computer executing an algorithm for controlling the DC current applied to three metallic needles (0.1 mm diameter and 20 mm length) separated by 20 mm distance and placed vertically over the sample holder, in which BIFIDO samples were placed.

The samples were subjected to an external electric field intensity ranging between 1.5 kV/mm to 6.6 kV/mm, while the current was automatically adjusted (AA) from the power supply.

The effect of applied polarity, voltage, gas, distance between the needle tip and the sample holder, the addition of maltodextrin as excipient and temperature in the chamber, were investigated in the EHD drying of BIFIDO. The conditions tested are summarized in Table 2.

TABLE 2
Test parameters

| | | | | | Distance | | | |
|---|---|---|---|---|---|---|---|---|
| | | | Current | | needles - | | | |
| Sample | Polarity | Voltage | intensity | Gas | sample | Number of | Maltodextrin | Temp. |
| designation | (+/−) | (kV) | (uA) | type | (mm) | needles | (% w/v) | (deg C.) |
| R0 | 0 | 0 | 0 | N2 | — | 0 | — | 20 |
| S1 | − | 3.7 | AA | N2 | 15 | 3 | — | 20 |
| S2 | + | 3.7 | AA | N2 | 15 | 3 | — | 20 |
| S3 | − | 4.7 | AA | N2 | 25 | 3 | — | 20 |
| S4 | + | 4.7 | AA | N2 | 25 | 3 | — | 20 |
| S5 | − | 10 | AA | Air | 15 | 3 | — | 20 |
| S6 | + | 10 | AA | Air | 15 | 3 | — | 20 |
| S7 | − | 3.7 | AA | N2 | 25 | 3 | — | 20 |
| S8 | − | 3.7 | AA | Air | 15 | 3 | — | 20 |
| S9 | − | 10 | AA | Air | 25 | 3 | — | 20 |
| S11 | − | AA | 200 | N2 | 15 | 3 | — | 20 |
| R13 | 0 | 0 | 0 | N2 | — | 0 | — | 30 |
| S14 | − | AA | 200 | N2 | 15 | 3 | — | 30 |
| R15 | 0 | 0 | 200 | N2 | — | 0 | — | 40 |
| S16 | − | AA | 200 | N2 | 15 | 3 | — | 40 |
| MF2 | − | 1.5 | 200 | N2 | 15 | 3 | — | 20 |
| MF3 | − | 1.5 | 200 | N2 | 20 | 3 | — | 20 |
| MF4 | − | 1.5 | 200 | N2 | 15 | 1 | — | 20 |
| MF5 | − | 1.5 | 200 | N2 | 20 | 1 | — | 20 |
| SM1 | − | 3.7 | AA | N2 | 15 | 3 | 40 | 20 |
| FD | 0 | 0 | 0 | Vacuum | — | 0 | — | −20 |
| FDM | 0 | 0 | 0 | Vacuum | — | 0 | 40 | −20 |

“Setup 2” was developed to optimize “Setup 1”. A moving compartment (M) was utilized, where a nylon filter membrane (MF) pore size 0.45 μm, diameter 90 mm (Sigma-Aldrich, Steinheim, Germany) was placed on a metallic mesh, the sample holder. The current intensity was constant at 200 μalways utilizing the computer algorithm, while the voltage was automatically adjusted (AA). The effect of needle number and distance between the needle tip and the sample holder were tested (samples MF2-MF5 described in Table 2).

For both setups 1 and 2, BIFIDO samples were subjected to the external electric field (EHD dried-S) or not (reference samples-R) for 1 and 2 hours.

### Freeze-Drying

The probiotics (3×10¹¹ cells/gram) stored at −80° C., were thawed at room temperature and transferred (5 g) to an aluminum sample holder 55 mm×80 mm (same protocol as EHD drying). BIFIDO samples were frozen at −20° C. for 24 h and then freeze-dried for 2 h (1,030 mbar) using a bench-scale freeze dryer (Beta LMC 1-8, Chist, Germany), sample FDM (Table 2). The experiments were executed in duplicates.

### Sample Characterization

### Water Loss (%)

The initial weight of the samples and their subsequent weight after 1 and 2 hours of EHD drying were measured with a digital balance of 0.001 g accuracy (Mettler Toledo, Greifensee, Switzerland). The % water loss (WL) of samples drying the experiments was calculated based on the following equation:

$$\% Water loss = \frac{Wo - W}{Wo} \cdot 100$$

where Wo, W are the initial weight of sample and the weight of the sample after EHD drying respectively.

### Cell Viability (Flow Cytometry)

Dried probiotics were stained with the dual staining kit BacLight™ consisting of the fluorophores SYTO9 (Excitation/Emission: 485/498 nm) and propidium iodide (PI) (Excitation/Emission: 535/617 nm). A BD Accuri™ C6 Plus system (BD Biosciences, Franklin Lakes, NJ, USA) was utilized for the flow cytometric analysis of bacterial viability. All analyses were executed at low flow rate (14 μL/min) and fixed maximum volume of collected sample at 10 μL. The samples' bacterial concentration was adjusted, according to the instructions of the manufacturer, between approximately 1×10³ and 5×10⁶ cells/mL, and appropriate gates were defined for the SYTO9 and PI-stained bacteria to be sorted at FL1-versus-FL3 dot plot (BD-Biosciences, 2012). The experiments were executed in triplicates.

### FTIR

Infrared transmittance measurements were carried out with an FT-IR spectrometer (Nicolet iS50, Thermo Fisher Scientific, Waltham, MA, USA) at room temperature (25° C.). Spectra were recorded after 32 scans at a resolution of 4 cm⁻¹ in a wave number range of 4.000 to 400 cm⁻¹.

### Bacterial Cell Surface Charge

BIFIDO dried cells at a concentration of approximately 10⁸ cells/mL were suspended in 10 mM KH₂PO₄ (Sigma-Aldrich, Steinheim, Germany) and transferred to a capillary cell with gold plated beryllium/copper electrodes (DTS1070 cell, Malvern Panalytical Ltd, Malvern, UK). The electrophoretic mobility of the samples was measured using a Zeta-sizer (Malvern Panalytical Ltd, Malvern, UK) and converted to zeta potential (mV) using the Helmholtz-Schmoluchowski equation. Each sample was analyzed in quintuplicate and all measurements were carried out at 25° C.

### Bacterial Cell Surface Hydrophobicity

The hydrophobic/hydrophilic character of the surface of BIFIDO cells after EHD and Freeze drying was evaluated by the microbial adhesion to hexadecane (MATH) assay (Deepika et al. 2009). BIFIDO probiotic cells were suspended in 10 mM KH₂PO₄ until an absorbance (OD600)˜0.8. An equal volume of 2 mL of the bacterial cell suspension and hexadecane (Thermo Fisher Scientific, Waltham, MA, USA) were vortexed for 1 min and allowed to stand for 20 min until complete phase separation. The OD400 of the aqueous phase was carefully removed and measured.

The percentage of microbial adhesion to hexadecane (or % Hydrophobicity) was calculated utilizing the following equation:

$$\% Hydrophobicity = ( \frac{1 - A 1}{A 0} ) \times 100$$

where AO is the initial absorbance of the bacterial suspension and A1 is the absorbance after phase separation at 600 nm. Samples were prepared in triplicate.

### Stastistical Analysis

The results are presented as mean value±Standard Deviation (SD). In order to analyze the differences among the, a single factor ANOVA was utilized. Statistically significant different samples results were considered for p-values below 5% (p<0.05).

### Results

### EHD Setup 1

### The Effect of Polarity and Gas Type

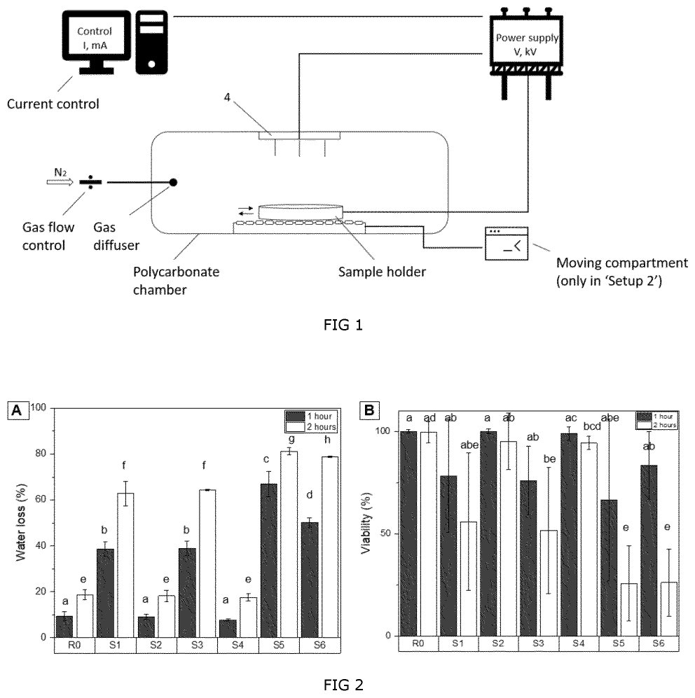

The effect of polarity on the EHD drying was investigated and is illustrated in FIG. 2. FIG. 2A shows that the water loss is an EHD drying parameter is time dependent, as after 2 h an increase in WL water loss (WL) was observed with time for all the samples.

Sample S1 (negative polarity) lost about 38.7±3.2% and 62.8±5.3% of water (statistically significant, P<0.05) after 1 h and 2 h, respectively, while S2 (positive polarity) lost about 9.1±1.3% and 18.2±2.4% of water (not statically significant) after 1 h and 2 h, respectively.

Sample S3 (WL=38.8±3.2% (1 h), WL=64.3±0.3% (2 h)), which was also dried under negative polarity lost higher amount of water (P<0.05) comparatively to sample dried with positive polarity S4 (WL=7.7±0.4% (1 h)), WL=17.5±1.6% (2 h)), respectively. Nevertheless, no significant differences were noted in terms of bacterial viability (V) when comparing samples dried using either negative polarity (sample S1 and S3), or positive polarity (sample S2 and S4).

It has been demonstrated that the negative corona creates a higher corona current than positive corona for the same voltage (Lai and Wong 2003), which could justify the greater water loss noticed for samples dried with negative polarity (FIG. 2). Moreover, the ionic wind produced at negative coronas presents higher velocities than for positive coronas (S. Chen, Van Den Berg, and Nijdam 2018) at low voltages (less than 10 kV/mm), which could have also affected the water evaporated from the EHD dried samples. The lower dielectric break-down strength of air (1.15 times lower than nitrogen) allowed the increase of voltage to 10 kV for samples S5 and S6. The sample S5, which was dried under the influence of negative corona discharge, also displayed significantly higher water loss (WL=67±5.4% (1 h), WL=81.3±1.6% (2 h)) and similar probiotic viability (V=66.3±39.6 (1 h), V=25.7±18.3% (2 h)) compared to sample S6 (WL=9.3±1.9% (1 h), WL=18.7±2.2% (2 h) and V=83.3±16.6% (1 h), V=26.1±16.3 (2 h)), which was dried under the influence of positive corona discharge. Herein, the higher contribution of the negative polarity comparatively to the positive polarity, to the EHD drying, was confirmed when either nitrogen or air was the surrounding gas. Samples dried using positive polarity presented similar water loss comparatively to the reference sample (R0), (almost no drying), highlighting the higher efficiency of EHD drying using negative polarity.

### The Effect of Voltage

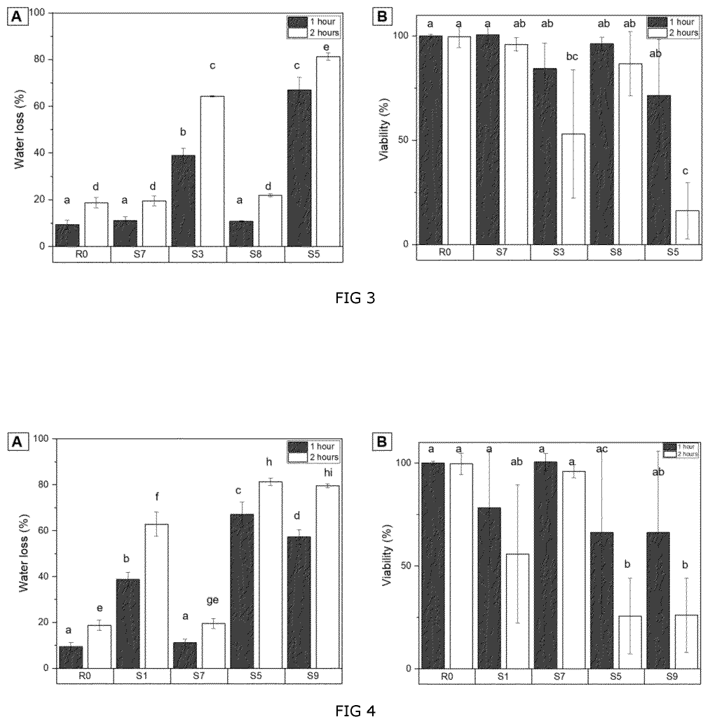

FIG. 3A demonstrates that an increase in the voltage from 3.7 to 4.7 kV, increased the water loss, after 1 h of drying, from 11.1±1.6% to 38.9±3.2% for samples S7 and S3, respectively. After 2 h of drying, the samples S7 and S3 lost about 19.5±2.1% and 64.32±0.3% of water, respectively. Similar trend was noted for the samples S8 (3.7 kV) and S5 (10 kV), where samples lost about 10.7±0.3% (1 h) and 21.9±0.6% (2 h) for sample S8, while for sample S5 the water loss was about 67±5.4% (1 h) and 81.3±1.6% (2 h). It has been demonstrated that an increase in the applied voltage strengthens the electric field by increasing the charge density current and velocity of the ionic wind, thus enhancing the drying rate (Anukiruthika, Moses, and Anandharamakrishnan 2021; Defraeye and Martynenko 2019; S. Chen, Nobelen, and Nijdam 2017).

No significant differences in the bacterial viability (FIG. 3B) were noted between the samples S7 (3.7 kV), S3 (4.7 kV) and the reference sample R0, when nitrogen was the surrounding gas. For sample S7, the probiotics viability decreased from 99.9±4.1% (1 h) to 95.9±3.2% (2 h), while in sample S3 the % of viable cells decreased from 84.4±12.1% (1 h) to 53±30.8% (2 h). The reference sample denoted a viability of approximately 99% for both tested hours.

When comparing samples S8 (3.7 kV), S5 (10 kV) to the reference sample (R0), significant differences on the % of viable probiotics were found. For sample S8, the viability decreased from 96.3±3.0% (1 h) to 86.6±15.3% (2 h), whilst for sample S5, the viability decreased from 71.5±26.8% (1 h) to 16.2±13.4% (2 h). Sample S5 has the highest evaporation rate among the samples tested in FIG. 3 and thus lower viability, contrarily to sample S7 and S8 that barely were dried.

Similar to other drying techniques, during EHD drying, the bacteria are subjected to dehydration and osmotic stress when water is removed from the cells and their surrounding environment. Therefore, it could be expected that increasing voltage would enhance water evaporation and decrease the bacterial viability.

The effect of the distance between the needles and the sample holder

The effect of distance between the high-voltage electrodes on each pair of samples is shown in FIG. 4. It was noticed that the sample S7 with a distance of 25 mm presented no significant differences with the reference R0 in terms of water loss (WL=11.1±1.6% (1 h), WL=19.5±2.1% (2 h)), while S1 with a distance of 15 mm had 3 times more water loss than S7 and R0 after two hours of drying (WL=38.7±3.2% (1 h), WL=62.8±5.3% (2 h)). The bacterial viability, though, was not statistically significant for both samples S7 (V=99.9±4.1% (1 h), and V=95.9±3.2% (2 h)) and S1 (V=78.3±27.6% (1 h), V=55.8±33.6% (2 h)), denoting no statistical difference with the reference sample (R0).

The samples S5 (15 mm) and S9 (25 mm) (dried under air conditions) presented statistically significant differences regarding their water loss for the first hour (WL=67.0±5.4% (S5), WL=57.3±3.2% (S9)), but not for the second hour of EHD drying (WL=81.0±1.6% for (S5), WL=79.6±0.9% (S9). Most of the water evaporation took place the first hour, while during the second hour of EHD drying the bacterial dehydration of S5 (V=66.4±39.6% (1 h), V=25.7±18.3% (2 h)) and S9 (V=66.3±39.5% (1 h), V=26.1±18.1% (2 h)) reached to a critical point where the viability was significantly decreased compared to the reference sample R0.

Similar to the increase of voltage, the decrease of the electrodes' distance is one more parameter affecting the corona discharge, forming a large ion current and increasing the ionic wind velocity, therefore accelerating the drying rates (Tirtha R. Bajgai et al. 2006). The present results indicated that the decrease of electrode spacing inversely affected the moisture removal from the samples.

### The Effect of Temperature

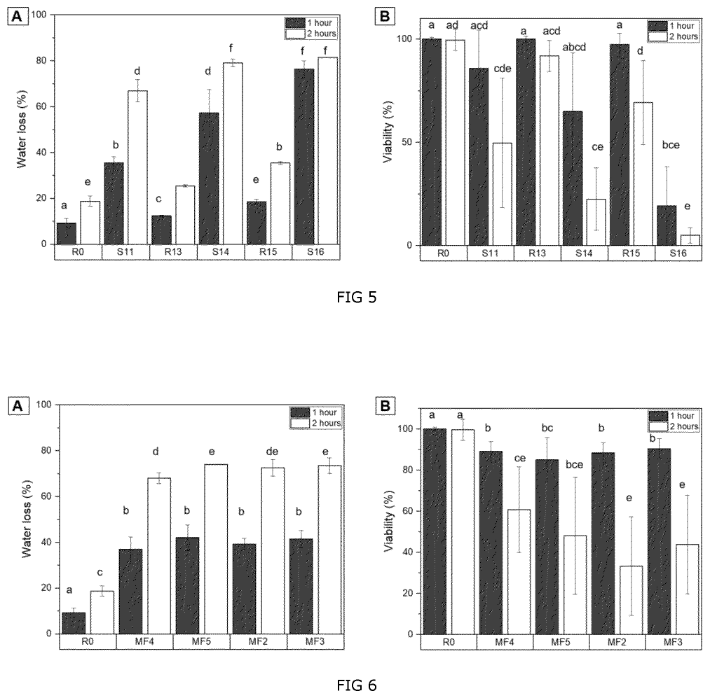

The effect of nitrogen temperature on the EHD drying of the samples S11, S14 and S16 is presented in (FIG. 5). The increase in nitrogen temperature from 20° C. to 30° C. and 40°° C. increased the water loss of S11 (WL=35.4±2.7% (1 h)), S14 (57.2±10.2% (1 h)) and S16 (76.3±3.6% (1 h)) respectively, while their corresponding viability decreased (V=85.9±18.5% (1 h), V=64.9±28.5% (1 h) and V=19.3±19% (1 h)).

The combination of EHD drying and heated nitrogen at 30° C. of sample S14 on the probiotic viability, did not present any statistically significant differences compared to S11 that was dried under the same electric field intensity and ambient temperature (20° C.). The water evaporation of S14, though, reached from the first hour of drying the same loss as S11 in after two hours (WL=66.9±4.8% (1 h)). Therefore, it can be concluded, that coupling EHD drying with elevated gas temperature could increase the water loss, due to convective and thermo-diffusive effects from the core to the surface of the samples, without compromising the bacterial viability at least for the first hour of drying. The sample S16 presented accelerated water loss from the first hour of EHD drying at 40° C. It can be noticed though, that the drying rate was decreased denoting no significant differences between the water loss after one (WL=76.3±3.6% (1 h)) and two (WL=81.4±0.1% (2 h)) hours of drying. It can be clearly seen, however, that such fast water loss markedly decreased the probiotic viability (V=19.3±19% (1 h), V=4.9±3.7% (2 h)).

### EHD Setup 2

### Effect of Needle Number and Spacing Between Needles and Sample Holder

To improve probiotics drying, a moving compartment was added for the periodical movement of the flat electrode (Setup 2). Furthermore, a mesh electrode and a nylon-filter were utilized, (under the probiotics) to facilitate the airflow passage (Mujumdar and Xiao 2019; Iranshahi, Martynenko, and Defraeye 2020).

The effect of the number of needles and the distance between the needles and the samples were also investigated and the results are illustrated in (FIG. 6). It was observed that neither the number of needles nor the electrode gap led to significant differences among the samples' MF4 (WL=37±5.3% (1 h), WL=68±2.3% (2 h)), MF5 (WL=42.9±5.6% (1 h), WL=74±0.1% (2 h)), MF2 (WL=39.3±2.5% (1 h), WL=72.5±3.5% (2 h)) and MF3 (WL=41.5±3.8% (1 h), WL=73.5±3.4 (2 h)) water loss and neither to the corresponding probiotics' viability (for MF4 V=89.2±4.8% (1 h), V=60.8±20.8% (2 h), for MF5 V=84.9±10.8% (1 h), V=48.1±28.5% (2 h), for MF2 V=88.4±4.9% (1 h), V=33.3±23.9% (2 h), for MF3 V=90.5±4.9% (1 h), V=43.8±23.9% (2 h)). Despite that fact, significant differences were noted among all the tested samples and the reference sample R0. The current experimental setup did not exhibit significant differences by increasing the number of needles and electrode gap, thus the sample MF4 was chosen for the further studies.

### Comparison of EHD With Freeze-Dryer

### Water Loss and Viability

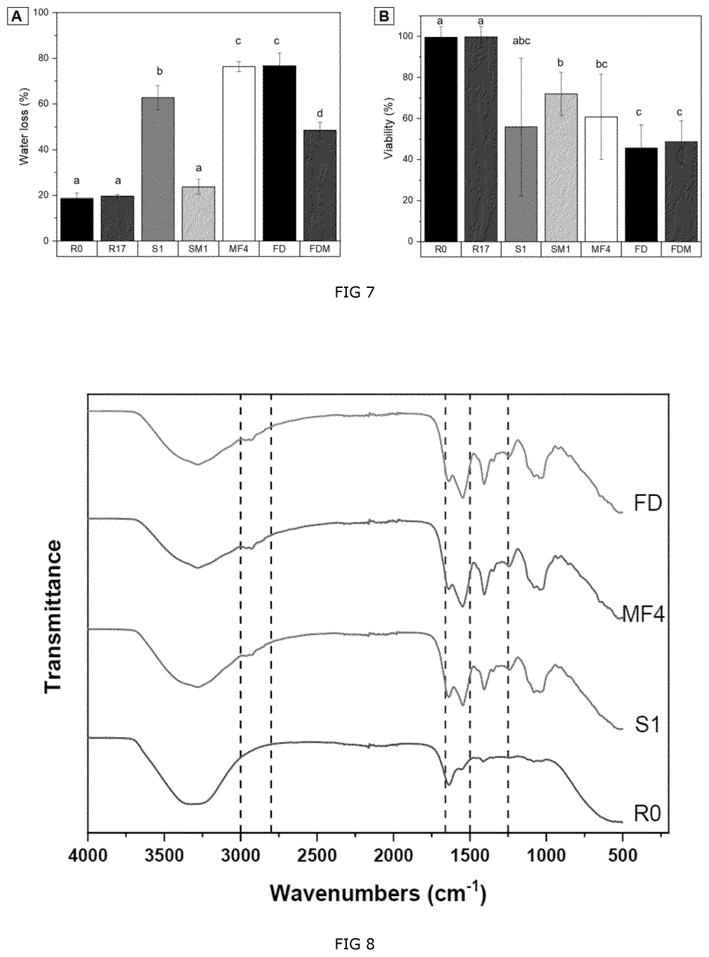

The effect of the employed two-hour drying process on the water loss and viability of probiotic bacteria is illustrated in (FIG. 7). Overall, the EHD dried sample MF4 with the optimized “Setup 2” presented the highest water loss (WL=76.4±2.2% (2 h)) along with the freeze-died sample FD (WL=76.8±5.5% (2 h)), while no significant differences were noted between them in terms of bacterial viability (for MF4 V=60.8±20.8% (2 h), for FD V=45.5±11.3% (2 h)).

The water loss of both MF4 and FD was the highest noted among the dried samples, while. sample S1 followed with a lower water loss of 62.8±5.3% (2 h)). The EHD-dried samples S1 and MF4 presented statistically different water loss results, implying that the filter-mesh electrode indeed facilitated the airflow and enhanced the water evaporation (Mujumdar and Xiao 2019; Iranshahi, Martynenko, and Defraeye 2020).

The addition of maltodextrin decreased the water loss of SM1 (WL=23.7±3.3% (2 h)) and FDM (WL=48.5±3.5& (2 h)) compared to the corresponding MF4 or FD samples without maltodextrin. Using single drop convective drying experiments of with different sugars, Adhikari (Adhikari et al. 2004) suggested that the addition of maltodextrin lowered their drying rate, due to the difficulty of water to diffuse though maltodextrin molecules. Similarly, Gianfrancesco et al also reported the decreased water flux towards the surface of the amorphous maltodextrin matrices, limiting the evaporation rate and water loss during the selected drying time (Gianfrancesco et al. 2012).

Nevertheless, SM1 (V=72±10.6% (2 h)) presented enhanced viability in comparison to both freeze-dried samples. Protective sugars, like maltodextrin, are known to depress the membrane phase transition temperature (Tm) and maintain the membrane integrity of the bacterial cells while being dehydrated (Mille et al. 2004; Strasser et al. 2009). Despite that fact, and no statistical differences between SM1 and S1 (V=55.8±33.6% (2 h)) were noted. Similarly, probiotics' viability for sample without maltodextrin FD (V=45.5±11.3% (2 h)) and sample with maltodextrin FDM (V=48.7±10.2% (2 h)) did not present significant differences.

### Fourier Transform Infrared Spectroscopy

Fourier transform infrared spectroscopy (FTIR) spectra of BIFIDO (FIG. 8) shows the main characteristic bands of probiotics cells. The band at ˜3280 cm⁻¹, which represents the stretching of the hydroxyl-group bond (—OH), appeared more intense for the non-dried BIFIDO, since that sample was not as dry are the samples treated with electrohydrodynamics or freeze-drying.

The fatty-acid region between 3000 and 2800 cm⁻¹ was not present in the spectrum of nondried bacteria, nonetheless it appeared for all dried samples. More specifically, the bands at 2966, 2936 and 2876 cm⁻¹ represent the asymmetric CH3 stretching, the asymmetric CH2 stretching and the symmetric CH3 stretching of the nonpolar site of phospholipid bilayer respectively were revealed after evaporating water from the cells (Santos et al. 2015; Shakirova et al. 2010; Dianawati, Mishra, and Shaha 2012). The aforementioned groups were revealed after evaporating the extra water that was present in the fresh probiotics and were therefore present in the fatty-acid region.

The transmittance of the bacterial protein region befalls occurs at frequencies between 1660-1500 cm⁻¹. More precisely, the carbonyl stretching of secondary amides (Amide I) was found at 1639 cm⁻¹ and a wide peak found at 1545 cm⁻¹ indicated the N—H bending of Amide II. There is no apparent evidence that the secondary structure of proteins was affected by the application of EHD or freeze-drying processes, as no shifts for Amide II were noticed. It should be noted though, that the peak at 1639 cm⁻¹ was more intense for the fresh probiotics, while the peak at 1545 cm⁻¹ was more intense for the freeze-dried sample FD and the EHD-dried MF4. The EHD dried sample S1 presented similar intensity of both peaks. Based on Bozkurt et al., increased contact of BIFIDO cells with water molecules would increase the Amide I absorption band, as observed in the current results (Bozkurt et al. 2019). Interestingly, the intensity of the Amide II adsorption could also be correlated with the water loss of the samples, since it appeared more intense for the more dried FD and MF4, less intense for the S1 and much reduced for the fresh BIFIDO. The vibration of P═O as of PO2-couldbe identified at 1250 cm⁻¹, while a wide carbohydrate and phosphate band was observed at frequencies 1100-980 cm⁻¹ for all samples. The maximum transmittance was noted at 1070 cm⁻¹ and 1040 cm⁻¹ due to the valence of C—O—C group vibrations of the polar site of phospholipid bilayers. The peaks at 1070 cm⁻¹ and 1040 cm⁻¹ decreased for the fresh bacteria due to extended contact with water molecules, as indicated by Bozkurt et al (Bozkurt et al. 2019).

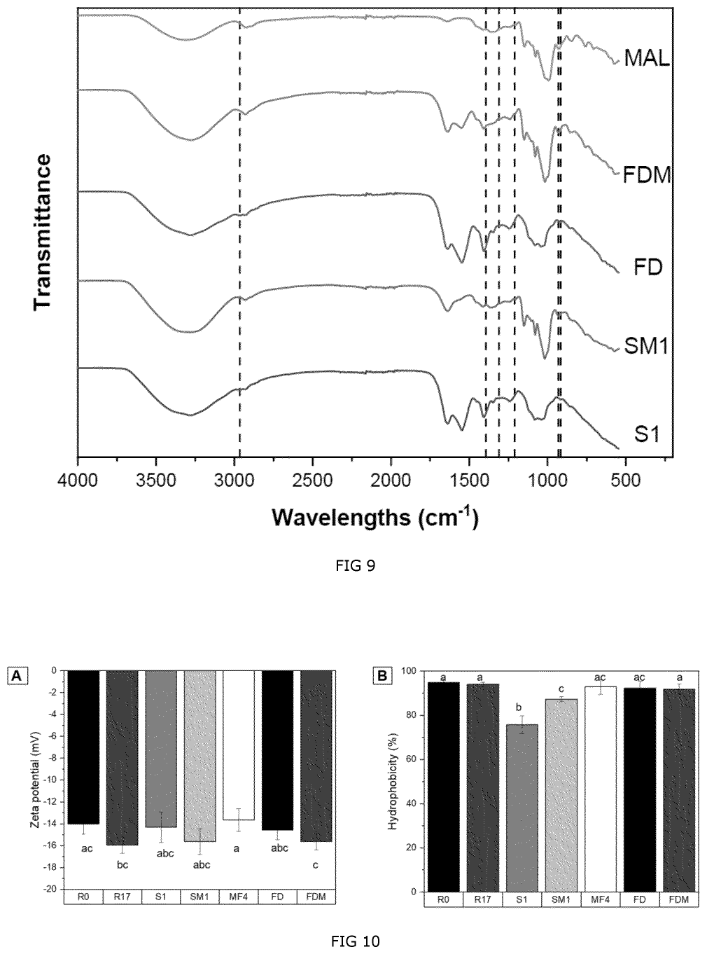

A comparison between EHD and freeze-dried samples with and without maltodextrin is presented in (FIG. 9).

The samples containing maltodextrin did not present the peak at 2966 cm⁻¹ and the intensity of the carbonyl stretching of secondary amides (Amide I) at 1639 cm⁻¹ was more intense than the N—H bending of Amide II at 1548 cm⁻¹. The above could be attributed to the fact the samples containing maltodextrin were characterized by a higher water content and increased contact of BIFIDO with water molecules could increase the amide I absorption band similar to the fresh BIFIDO samples. The wider peak at 1394-1310 cm⁻¹ is attributed to the chemical structure of maltodextrin, as well as the additional small peak at 1210 cm⁻¹ and a relatively intense peak at 1150 cm⁻¹. The latter peaks are representing C—O—C, C—O bands dominated by ring vibrations of carbohydrates. Moreover, an interaction between PO2 of bacterial envelopes and maltodextrin is implied by the alteration to lower frequency from 1040 cm⁻¹ for dried BIFIDO to 1020 cm⁻¹ for samples with BIFIDO and maltodextrin. The latter can be confirmed by noticing the shift of maltodextrin band from 995 to 1020 cm⁻¹. Such interactions, though hydrogen bond, of the phospholipid site of cell lipids with sugars has been previously suggested and described in literature (Oldenhof et al. 2005; Leslie et al. 1995; Dzuba, Leonov, and Surovtsev 2020). FTIR technique has been extensively utilized in the investigation of the protectant effects of sugars, such as maltodextrins. It should be mentioned, though, that the protective effect of such high molecular weight molecules is indirect, by facilitating the replacement of cell surface water from smaller sugars, such as glucose (Santos et al. 2015).

### Zeta-Potential

The bacteria were electronegative in all samples, as it has been suggested previously for other Bifidobacterium strains (Gómez Zavaglia et al. 2002). Such negative cell surface charges at neutral pH could designate the dominance of anionic domains such as strong acids (phosphate based (lipo-) teichoic acids) and weak acids (acidic polysaccharides and proteins) (Deepika et al. 2009).

According to the FIG. 10A, no significant differences were noted among the zeta potential values of non-dried and dried samples. Sample R0 as well as EHD or freeze-dried samples that did not contain maltodextrin (S1 and FD) presented lower absolute values of zeta-potential compared to the corresponding samples containing maltodextrin (R17, SM1 and FDM). The latter results could confirm the indications from the FTIR spectra that maltodextrin molecules have interacted with the bacterial membrane surface. When the absolute zeta potential value increases in colloidal systems, more stable clusters are formed during electrophoresis. It could therefore be assumed that the interaction of sugars with the bacterial surface could stabilize the surface of the bacteria (Kłodzińska et al. 2010).

### Hydrophobicity

The microbial adhesion to hydrocarbons test has been commonly utilized for the evaluation of cell surface hydrophobicity in Bifidobacteria (Pelletier et al. 1997; Gómez Zavaglia et al. 2002). When bacteria are hydrophobic, they present a high adherence (%) to hexadecane. As shown in FIG. 10B, all the samples presented similar values of hydrophobicity (FIG. 10B). Interestingly, the samples EHD dried samples with setup 1, presented statistically different results with lower hydrophobicity compared to the EHD 2, control and the freeze-dried samples.

Moreover, the addition of maltodextrin in the same sample increased the hydrophobicity of BIFIDO. According to Shakirova et al. (2010) the cell surface hydrophobicity was associated with higher content of BIFIDO surface proteins (Shakirova et al. 2010). Many other studies have suggested that the presence of (glycol-) proteinaceous material and polysaccharides to be responsible for higher hydrophobicity and hydrophilicity respectively (Collado, Meriluoto, and Salminen 2008; Boonaert and Rouxhet 2000; Van Der Mei et al. 2003). Therefore, as indicated by both the electronegative character of BIFIDO and the high hydrophobicity, a protein-rich bacterial surface is suggested, as also seen in FTIR spectra.

### Conclusion

Electrohydrodynamic drying can be used to dry probiotic cells. The EHD drying process was found to be dependent on several parameters such as polarity and voltage of the ionic wind, collector type, surrounding temperature and gas, probiotic concentration, and use of excipient. The optimized conditions using the Setup 1 allowed to reach 70% of drying and cell viability up to 34%. Probiotics viability could be increased up to 50% after 2 hours, by dispersing probiotics with maltodextrin. However, the addition maltodextrin decreased the water evaporation rate by 30%. The EHD performance (cell viability and water loss) was improved using a moving mesh flat electrode under the probiotics to facilitate the airflow of the drying, called Setup 2. Using this Setup 2, the water evaporation and the cell viability of the probiotics were increased up to 78% and 70%, respectively, comparatively to Setup 1. Moreover, comparison of the EHD drying with freeze drying processes, reveals that the survival of probiotics and the water evaporation rates were similar, while the cell surface properties remained similar to the non-dried cells.

### Example 2

### Materials and Methods

Probiotic cells Bifidobacterium animalis, DSM 15954 (in the following called BIFIDO) were provided by Chr-Hansen A/S (Hørsholm, Denmark). Maltodextrin of Dextrose Equivalent 12 (Glucidex IT12, Roquette, Lestrem, France) was used for the encapsulation of BIFIDO. Sodium chloride (NaCl) (Merck, Kenilworth, NJ, USA) and tryptone (Oxoid, Hampshire, UK) were used for the preparation of isotonic solutions. KH₂PO₄ (VWR International, Leuven, Belgium) was utilized as suspending medium for the evaluation of the bacterial surface properties and hexadecane (Thermo Fisher Scientific, Waltham, MA, USA) for carrying out the hydrophobicity measurements. The bacterial viability was performed using the dual staining LIVE/DEAD™ BacLight™ Bacterial Viability Kit (L-7012, Molecular Probes, Eugene, OR, USA). Fluorescent dye Thiazole Orange, dye content˜90% (Sigma-Aldrich, USA) was employed for the staining of the BIFIDO cells.

### Evaluation of Bacterial Cell Surface Physicochemical Properties

### Bacterial Cell Surface Electrical Charge

BIFIFO probiotic cells at a concentration of approximately 3*10⁸ cells/mL were suspended in 10 mM KH₂PO₄ to obtain an optical density (OD600)˜1.0. The pH of solutions was adjusted to 1, 2, 4, 6 and 8 with 1 M HCl or 1 M NaOH. The electrophoretic mobility was determined using a Zeta-sizer (Malvern Panalytical Ltd, Malvern, UK). A volume of 1 mL of sample was injected into a folded capillary cell with gold plated beryllium/copper electrodes (DTS1070 cell, Malvern Panalytical Ltd, Malvern, UK). The measurements were carried out at 25° C. and each sample was analyzed in quintuplicate. The electrophoretic mobilities were converted to zeta potential using the Helmholtz-Schmoluchowski equation.

### Bacterial Cell Surface Hydrophobic/Hydrophobic Character

The microbial adhesion to hexadecane (MATH) assay was employed to evaluate the hydrophobic character of the surface of BIFIDO cells at different pH (1, 2, 4, 6, 8). The probiotic cells were suspended in 10 mM KH₂PO₄ to obtain an optical density (OD400)˜0.8. Two milliliters of the bacterial cell suspension were mixed with equal volume of hexadecane, vortexed for 1 min and were allowed to stand for 20 min to ensure complete phase separation. The aqueous phase was carefully removed after equilibration and the OD400 was measured.

The percentage of microbial adhesion to hexadecane was calculated utilizing the following equation:

$$\% Adhesion = ( \frac{1 - A 1}{A 0} ) \times 100$$

Where A0 is the initial absorbance of the bacterial suspension and A1 is the absorbance after 20 min of incubation and phase separation (OD400).

Each measurement was performed in triplicate.

### Electrospray of Probiotic Solutions

The maltodextrin (75% w/v) was dispersed in Millipore water and stirred until obtaining a homogeneous solution, then BIFIDO probiotic cells (10% v/v) were added and stirred until they were fully dispersed.

The electrospray set-up included a high voltage generator (ES50P-10W, Gamma High Voltage Research, Inc., USA) to provide a voltage of 15-40 kV, and syringe pump (New Era Pump Systems, Inc., Farmingdale, NY, USA) to feed the maltodextrin/BIFIDO dispersion at a flow rate of 0.03 mL/min The Maltodextrin BIFIDO capsules were collected horizontally on a steel plate covered with aluminum foil placed at a distance of 10 cm from the end of the needle (27 Gouge, 12 mm length, 0.21 mm inner diameter). The electrospray set-up was placed inside a chamber with nitrogen flow, and the temperature and relative humidity were 23° C. and 22±3% respectively.

The polarity of the electrode connected at the needle tip of the syringe was positive or negative, then as a matter of brevity, the sample is referred to as electrosprayed under positive or negative polarity respectively. Moreover, the charge applied on the needle tip is denoted first, and the charge applied on the collector. For example, when −15 kV was applied on the nozzle tip and +5kV on the collector, then it is referred as (−15 kV) (+5 kV), and the absolute value of the electric potential is presented as |20 kV|.

### Confocal Laser Scanning Microscope

The Inverted Zeiss LSM-710 Confocal laser scanning microscope (Carl Zeiss MicroImaging GmbH, Jena, Germany) equipped with a diode laser (405 nm), argon laser (458, 488 and 514 nm), two HeNe lasers (543 and 633 nm), and three detectors and one transmitted detector, was utilized. The probiotic cells were stained with the fluorescent dye Thiazole Orange, dye content˜90% (Sigma-Aldrich, USA) and washed with NaCl 0.85% w/v prior to encapsulation. The dye was in powder format, therefore it was dissolved in dimethyl sulfoxide (DMSO) at concentration 42 μmol/L. For image acquisition, the samples were scanned by using the Z-series mode, and confocal fluorescence pictures were taken with 20×, 40× and 60× objectives. All images were acquired at an excitation wavelength of 488 nm and emission bandpass filter between 505-550 nm. The images were analyzed using the ZEN software.

### Fourier Transform Infrared Spectroscopy

The Fourier transform infrared spectroscopy (FTIR) spectra of the samples were recorded using a Nicolet iS50 spectrometer (Thermo Scientific, NY, USA) in attenuated total reflection (ATR) mode over the range of 400-4000 cm⁻¹. All spectra were recorded in transmission mode, with a scanning resolution of 4 cm⁻¹ and 32 scans, and at room temperature (25° C.). Spectral analysis was carried out using Omnic software (Thermo).

BIFIDO pure culture (no use of maltodextrin as encapsulation compound) was electrosprayed with negative (−15 kV) (+5 kV) and positive (+15 kV) (−5 kV) polarities, and 200 μL of the electrosprayed samples were assessed by ATR-FTIR. Same volume of BIFIDO not treated with an external electric field, was also assessed, as control.

### Differential Scanning Calorimetry

Differential scanning calorimetry (DSC) experiments were performed to determine the glass transition temperature (Tg) of the electrosprayed microcapsules. The thermograms of the samples were obtained using the DSC 250 (TA Instruments, New Castle, Delaware, USA), equipped with Refrigerated Cooling System 90. The instrument was calibrated in terms of heat flow and temperature using distilled water (melting point (m.p.)=0 C; DHm=334 J/g) and indium (m.p.=156.5 C; DHm=28.5 J/g). Nitrogen was used as a carrier gas at a flow rate of 50 mL/min.

Approximately 5 mg of the electrosprayed microcapsules (immediately collected after processing) were placed in pre-weighted standard DSC aluminum pans (Tzero aluminum Hermetic pans, TA Instruments, USA) and were hermetically sealed. An empty hermetically sealed aluminum pan was used as reference. The pans were first equilibrated for 5 min at 10° C., after which a heating ramp followed with 3° C./min to 120° C. The settings of the run were set at least 30° C. below and above the expected Tg of the electrosprayed microcapsules, based on the Tg of maltodextrin DE12.

The obtained DSC thermograms were analyzed using Trios software interfaced with the DSC, and the Tg was determined from the onset, mid, and end points from the shift in the curve line. All measurements were done in triplicate and the Tg values were averaged.

### Stability of Microencapsulated BIFIDO During Storage

Microcapsules containing BIFIDO electrosprayed with negative (−15 kV) (+5 kV) and positive (+15 kV) (−5 kV) polarities and non-encapsulated Bifidobacterium animalis as a reference sample, were stored in an incubating cabinet (Model KB 8400F, A/S Ninolab, Solrød Strand, Denamrk) at a relative humidity of 35±2% RH at 25° C. The viability of the encapsulated and non-encapsulated BIFIDO cells as a function of time (0 to 14 days) was determined by Colony Forming Unit (CFU) analysis. In particular, the electrosprayed microcapsules were dispersed in 0.85% w/v sodium chloride (NaCl) and 0.1% w/v peptone (tryptone) solution, homogenized by vortexing and then 0.1 ml of appropriate decimal dilutions was spread-plated in de Man Rogosa Sharpe (MRS) agar plates supplemented with 0.05 w/V % L-cysteine hydrochloride monohydrate (Merck, Kenilworth, NJ, USA). The MRS agar plates were incubated at 37° C. under anaerobic conditions (AnaeroGen, Oxoid, Hampshire, UK) for 3 days and the cell viability was calculated as the average of 4 plates.

The Encapsulation Efficiency (EE) of the microcapsules was determined by defining the number of viable cells inside the capsules (N) divided by the number of viable cells in the initial solution (NO) as expressed using the following equation:

$$E E ( \% ) = \frac{Log N}{Log N 0} \times 100$$

### Statistical Analysis

The results are expressed as mean value±Standard Deviation (SD). A single factor ANOVA was used to analyze the differences in the samples that were each time compared. Statistically significant differences among the samples' results were considered for p-values below 5% (p<0.05).

### Results and Discussion

### Bacterial Cell Surface Electrical Charge and Cell Surface Hydrophobic/Hydrophilic Character

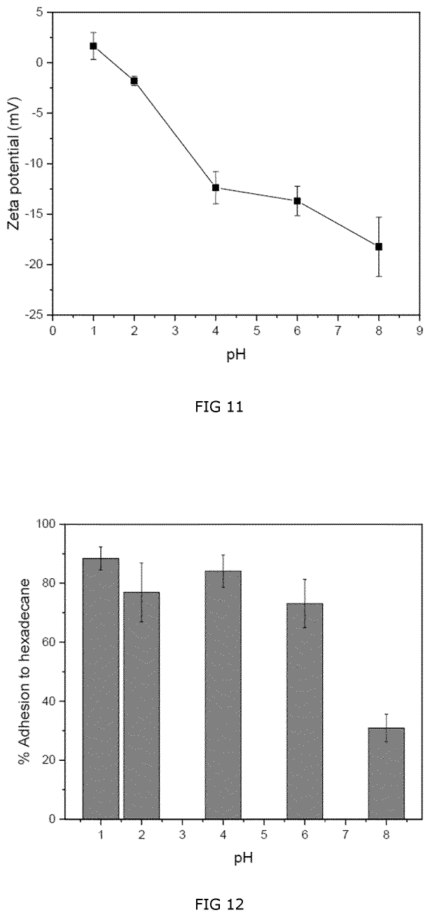

The zeta potential of BIFIDO in monopotassium phosphate buffer as function of pH is shown in FIG. 11. Cell surface electronegativity decreased with decreasing pH, showing negative zeta potential values above pH 2 and slightly positive values at pH 1. Similar zeta potential profiles have been previously reported for other Bifidobacterium strains (Bifidobacterium bifidum and Bifidobacterium pseudolongum) (Gómez Zavaglia et al., 2002), due to that the surface composition of the cells is dominated by anionic domains, including strong acids (phosphate based (lipo-)teichoic acids) and weak acids (acidic polysaccharides and surface proteins) (Deepika et al., 2009). The gradual protonation of the afore-mentioned chemical groups could justify the decreasing electronegativity of the bacteria for decreasing pH (Pelletier et al., 1997).

The hydrophobicity of the BIFIDO cell surface was evaluated by studying the adhesion of BIFIDO to hexadecane (FIG. 12). At pH values 1 to 6, BIFIDO showed a similar affinity to hexadecane (806.9%), suggesting a hydrophobic surface character. At higher pH values (pH=8), BIFIDO showed a lower hydrophobic character, with a percentage of affinity to the apolar hexadecane below 40%. Covalently bound proteins, S-layer proteins and fatty acids that are present at the surface of BIFIDO cells (Dianawati & Shah, 2011; Shakirova et al., 2010, 2013), have been suggested to promote the adherence to hydrophobic solvents and contribute to elevated bacterial hydrophobicity.

### Effect of the Electric Field Polarity and Voltage on the ‘Self-Organization’ of the Cells Within the Microcapsules

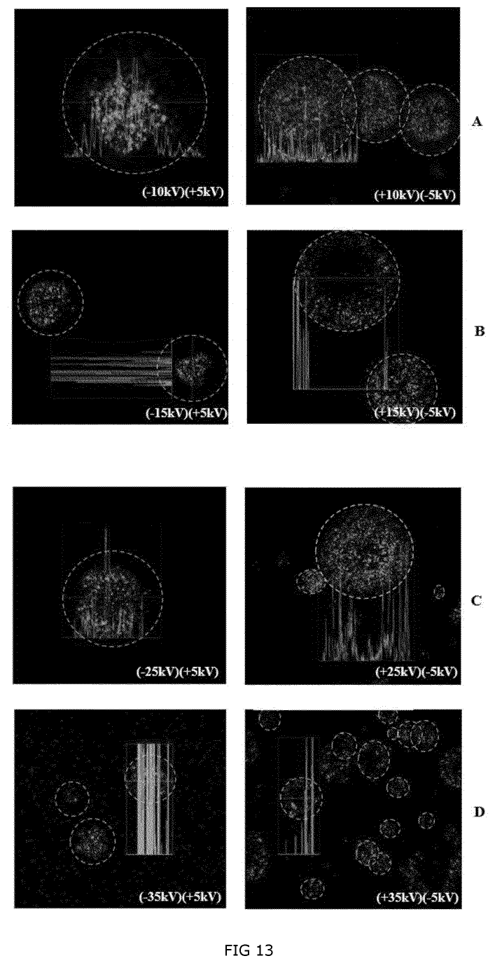

The effect of negative and positive polarity of the electric field on the distribution of probiotics cells within the electrosprayed maltodextrin—BIFIDO capsules at pH6, was studied by confocal laser scanning microscopy (CLSM) (FIG. 13).

Negative polarity at the needle resulted to a ‘self-organization’ of the probiotics cells mostly at the core of the electrosprayed capsules. This is due to that the negatively surface charged BIFIDO cells (at pH6) repulsed electrostatically by the negatively charged electric field at the needle walls, as well as at the Taylor cone inner surfaces. Analogous ‘self-organization’ of BIFIDO cells at the core of the electrosprayed capsules was observed by utilizing different voltages with negative polarity at the needle (FIG. 13). On the contrary, when a positive polarity was applied at the needle, the cells were distributed towards the outer surface of the electrosprayed capsules (FIG. 13).

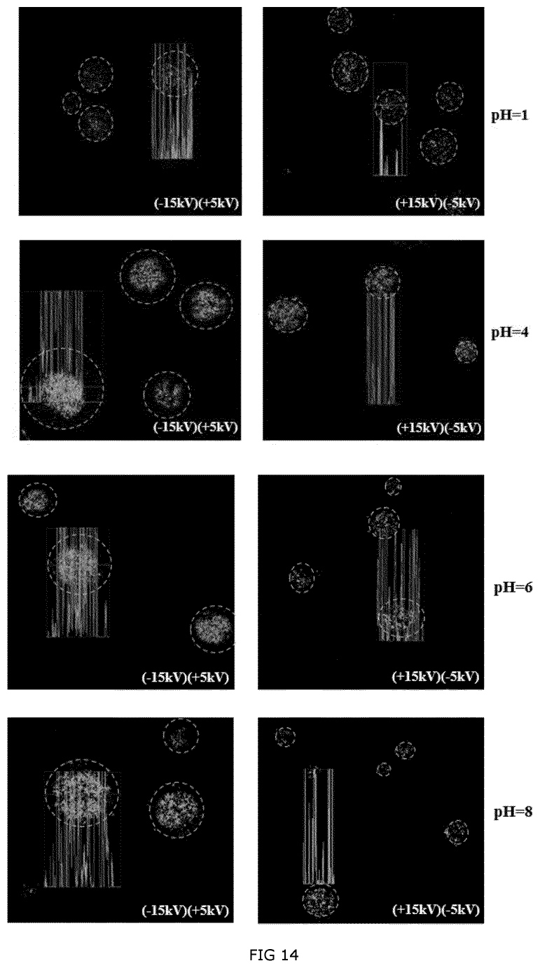

FIG. 14, shows the CLSM of the electrosprayed maltodextrin/BIFIDO dispersions at different pH values. Similarly to the results observed at pH 6, negatively surface charged BIFIDO cells at pH4-8 were ‘self-organized’ at the core of the capsules when negative polarity electrical field was applied at the needle, while cells at pH 1 (slightly positive surface charges) distributed closer to the outer surface of the capsules. Thus, the encapsulation of the BIFIDO at the core and outer surface of the capsules is due to the movement of the charged cells by Coulombic and electrophoretic forces within the DC electric field. Moreover, it is to note that as the probiotics exhibited similar hydrophobic character at pH 1 to 6 (FIG. 12), therefore the ‘self-organization’ of the cells within the electrosprayed capsules was generated due to their surface charge, rather than due to the cell's surface hydrophobic/hydrophilic character.

### Glass Transition Temperature of Electrosprayed Maltodextrin/PROBIO Capsules

The effect of voltage and polarity of the electrical field, on the glass transition temperature (Tg) of the maltodextrin-BIFIDO electrosprayed capsules was also assessed. The Tg is related to the moisture content of the electrosprayed capsules (Tg increases with decreasing the moisture content) as well as to the subsequent stability and viability of the encapsulated cells (Drake et al., 2018; Haque & Roos, 2004; Terpou et al., 2019). Removal of sufficient water and formation of non-crystalline glassy solid matrix is required in order to achieve stability during storage of the cells in the dried state (Drake et al., 2018).

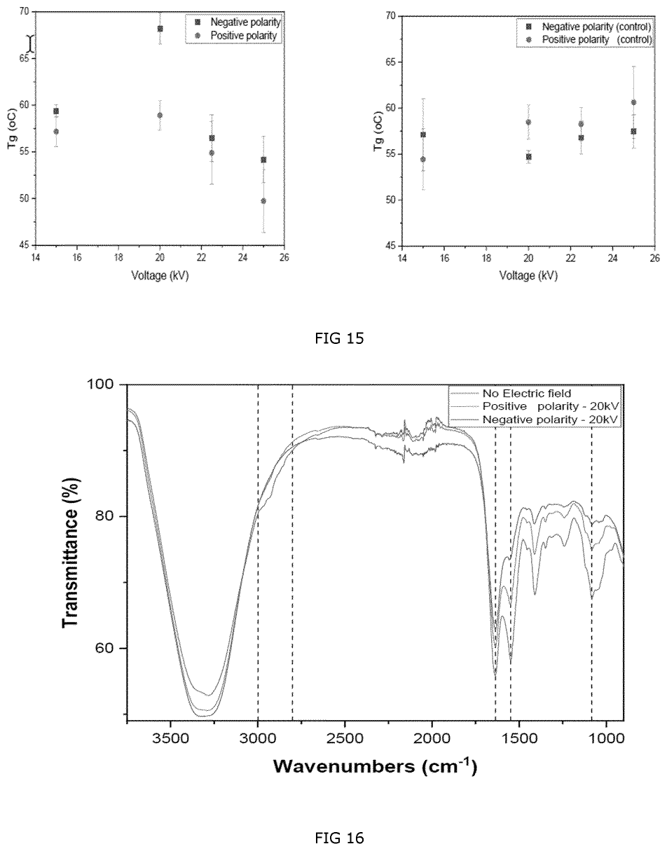

The glass transition temperature (Tg) of the electrosprayed maltodextrin capsules without probiotic cells changed slightly with increasing electrospray voltage, both for positive and negative polarity. Slightly higher Tg values of maltodextrin capsules were observed using the positive polarity electric field. On the contrary, the electrosprayed maltodextrin—BIFIDO capsules at pH 6 (FIG. 15) presented a maximum Tg value at |20 kV| ((−15 kV) (+5 kV)), and a subsequent decrease of the Tg with increasing electrospraying voltage. Moreover, the maltodextrin-BIFIDO capsules processed using a negative polarity were characterized by much higher Tg values than the capsules produced using a positive polarity. Particularly, the maltodextrin-BIFIDO capsules produced using a negative polarity of (−15 kV) (+5 kV) were characterized by a Tg of 68.21±1.66° C., a value which is was significantly higher to the other electrosprayed capsules processed using a negative polarity. When a positive polarity (+15 kV) (−5 kV) was used, the Tg was about 10° C. lower than the capsules produced using the same voltage of negative polarity (−15 kV) (+5 kV). As suggested above, at pH 6 the (negative surface charged) probiotic cells electrosprayed using a negative polarity electrical field were repelled and ‘self-organized’ in the core of the capsules, while the solvent accumulated near to the droplet surface. Thus, an enhanced evaporation of the solvent during electrospray initiated from the surface of highly charged droplets when a negative polarity electrical field was applied, in comparison when to the positive one. This enhanced evaporation leads to a lower moisture content for the negative polarity electrosprayed maltodextrin—BIFIDO capsules, and subsequent higher Tg values in comparison to the corresponding capsules processed using positive polarity. Thus, the different electrical field voltages and polarities affect the thermal properties and the glass transition of the electrosprayed capsules due to the presence of the surface charged probiotics.

### Fourier Transform Infrared Spectroscopy

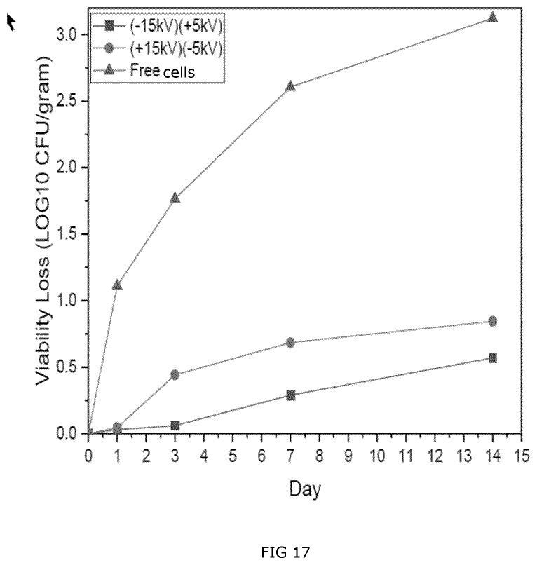

The FT-IR spectra of (non-encapsulated) electrosprayed BIFIDO (under the influence of different electric field polarity), as well as BIFIDO not treated with an external electric field, are presented in (FIG. 16). The band at ˜3280 cm⁻¹ represents the stretching of the hydroxyl-group bond (—OH) and is associated with the water related bonds of the samples. The band appeared more intense for the non-treated BIFIDO, while the lowest hydroxyl-group stretching intensity was noted for BIFIDO electrosprayed with negative polarity (−20 kV) (FIG. 17A). Increased —OH band intensity after FTIR measurements of spin-coated BIFIDO biofilms in water moisture environment has been previously reported in literature (Bozkurt et al., 2019).

Differences at the intensity of bands in the fatty-acid region between 3000 and 2800 cm⁻¹ were apparent between the differently treated samples, being more intense in the spectrum of the electrosprayed samples (FIG. 16). The bands at 2961, 2930 and 2876 cm⁻¹ signify the asymmetric CH3 and CH2 stretching and the symmetric CH3 stretching of the nonpolar site of phospholipid bilayer correspondingly (Dianawati et al., 2012; Santos et al., 2015; Shakirova et al., 2010). Higher transmittance intensity was observed for BIFIDO electrosprayed using negative polarity in comparison with the positive polarity electric field. The presence of water has been suggested to hinder the intensity of these groups (Dianawati et al., 2012), implying that less water was present in the bacteria electrosprayed at −20 kV, more water at +20 kV and even more in non-treated bacterial samples that their interferogram completely lacked of these peaks.

The main protein amide bands for biological samples are typically found at frequencies ˜3200 cm⁻¹ (Amide A), 1660-1500 cm⁻¹ (Amide I and Amide II), and 1310-1240 cm⁻¹ (Amide III) (Gautier et al., 2013; Krimm & Bandekart, 1986). For the non-electrosprayed BIFIDO, the carbonyl stretching vibration of the peptide bonds from secondary amides (at ˜1640 cm⁻¹, Amide I) was noticed to be more pronounced, in comparison to the Amide II peak at ˜1550 cm⁻¹. Nevertheless, for the electrosprayed BIFIDO, a less pronounced peak for Amide I in comparison to Amide II was observed, and this peak appeared to also be the least intense for the BIFIDO electrosprayed using negative polarity (FIG. 16). Bozkurt et al. studied the influence of relative humidity on the surface structure of spin-coated BIFIDO biofilms, concluding that the increased Amide I absorption is indicative of an increased contact of BIFIDO with water molecules (Bozkurt et al., 2019). Therefore, the decreased Amide I peak intensity of sample electrosprayed at negative polarity is suggestive for more dried samples.

Furthermore, differences at the intensity of bands at the polar site of the phospholipid bilayers of the cells (denoted at frequencies 1070 cm⁻¹ and 1040 cm⁻¹ due to the valence of C—O—C group vibrations) were also observed among the samples (FIG. 6). The decreased transmittance intensity for the non-electrosprayed BIFIDO is an indication of an extended contact of the cells with the water molecules, while the highest intensity found at the samples electrosprayed at negative polarity suggests that least water was present in BIFIDO electrosprayed using negative polarity, in comparison to the sample electrospared using positive (Bozkurt et al., 2019).

### Stability and Cell Viability of Microencapsulated Probiotics During Storage

As shown in FIG. 17, the probiotics encapsulated using negative polarity, presented a significantly higher viability (˜7.3 times higher, 0.06 log loss), compared to the encapsulated probiotics using positive polarity (0.44 log loss), even after 3 days of storage. Similar trend in viability (about 2.4 and 1.5 times higher) was monitored even after 7 and 14 days of stability studies respectively. Non encapsulated BIFIDO had 29.5, 9 and 5.5 times lower viability than cell encapsulated using negative polarity after 3, 7 and 14 days of storage. Moreover, it is to note, that the encapsulation efficiency of the microcapsules was similar for both polarities approximately 92% (92.9%±2.1 and 92.4%±1.2 electrosparyed with positive and negative polarities, respectively).

Thus, the encapsulated maltodextrin-BIFIDO electrosprayed using negative polarity, showed higher viability up to about 14 days of storage in comparison to the cells electrosprayed using positive polarity. This arises from the ‘self-organization’ of the cells in the core of the electrosprayed capsules using negative polarity, and the lower moisture content (and water activity) of these capsules, which certainly provide better stability and viability conditions for the encapsulated cells. Hence, the stability and the viability of the encapsulated probiotics can be effectively controlled utilizing the polarity of the external electric field.

### Conclusion

In the present study, the encapsulation of the Gram-positive bacteria Bifidobacterium lactis, DSM 15954, within maltodextrin capsules using both negative and positive charged electrospray needle, was investigated. The charge polarity of the needle of a DC electric field, directed the surface charged probiotic cells, either to the core or near the outer surface of the capsule, as documented by confocal microscopy images. This is a novel ‘self-organization’ process, resulting into the formation of different ‘composition’ of the core or outer surface of the microcapsules during electrospray processing. This can be further exploited to develop electrosprayed capsules with tailored core and outer surface compositions, based on the interplay of the applied electric field and charged molecules of the dispersions. Charge polarity applied to the electrospray needle controls not only the ‘self-organization’ of the probiotic cells within the liquid jet, but as well the mass transfer, the solvent evaporation and the physicochemical properties (e.g. glass transition) of the capsules.

The probiotics encapsulated using negative polarity, presented significantly higher glass transition values (nealy 10° C.) than the capsules produced using a positive polarity, and much greater viability (˜7.3 times higher), compared to the encapsulated probiotics using positive polarity, even after 3 days of storage at RH of 30% and 25° C.

The charge density on the bacterial surface was sufficient for their ‘self-organization’ when a low conductive, or neutral, maltodextrin matrix was employed. High charged density biopolymers (i.e., polyelectrolytes), that will change the charge density of the solution, could further functionalize the self-organization and distribution of the cells. The polarity of the electrical field could also be used to modify the molecular interactions between polyelectrolytes, and between polyelectrolytes and charged probiotic cells. Thus, it may be advantageous to use polyelectorlytes with the same charge as the cells, or with different charge compared to the cells, or with a neutral charge. An increase in the conductivity of the solution through the addition of a salt, that increases the surface charge density, can also be used to modulate the encapsulation of cells. In addition, by selecting probiotic cells having a sufficiently strong surface charge (at pH not close to their isoelectric point) will further contribute to selectively localization of the encapsulated cells.

Moreover, the present study essentially assess how to improve the protection and the viability of alive probiotic cells encapsulated by electrospray. The viability of the encapsulated cells substantially improved when the cells were located at the core of the capsules, as compared to case where the probiotics were distributed near the outer surface of the capsules.

1. A process for electrostatic spray drying of a living microorganism, the process comprising the following steps:
a) providing a suspension, wherein the suspension comprises a microorganism with an electrical surface charge and a formulation aid;
b) applying an electrostatic charge to said suspension;
c) forming droplets of said suspension;
d) drying said droplets, thereby forming dried particles; and
e) collecting the dried particles;
wherein the electrostatic charge has the same polarity as the electrical surface charge of the microorganism.

2. The process according to claim 1, wherein the electrostatic charged is applied to the suspension, while simultaneously moving the suspension.

3. The process according to claim 1, wherein the collecting of dried particles is achieved using a collector with the opposite charge compared to the electrostatic charge and the electrical surface charge of the microorganism.

4. The process according to claim 1, wherein the microorganism with a negative surface charge is selected from the group consisting of Bifidobacterium animalis, Lactobacillus acidophilus, Pseudomonas aeruginosa and Escherichia coli.

5. The process according to claim 4, wherein the microorganism is Bifidobacterium animalis subsp. lactis (DSM 15954).

6. The process according to claim 1, wherein the shape of dried particles is a sphere, fiber, spiral, rod or hybrid.

7. The process according to claim 1, wherein the formulation aid is a polysaccharide, protein, lipid or synthetic polymer, or a mix thereof.

8. The process according to claim 7, wherein the polysaccharides are either positively charged, negatively charged or neutral.

9. The process according to claim 8, wherein the polysaccharides are selected from the group consisting of maltodextrin, starch, xanthan, gellan, alginate, pectin, glucan and chitosan.

10. The process according to claim 7, wherein the proteins are dairy proteins, non-dairy animal proteins, plant proteins, algae proteins or fermentation product proteins.

11. (canceled)

12. A particle comprising a polymer and at least one cell of a living microorganism, wherein the at least one cell is located in a core of the particle, and wherein the polymer composition of the particle is uniform and forms a shell around the core that is substantially devoid of cell(s).

13. A particle comprising a living microorganism, obtained by a process according to claim 1.

14. (canceled)

15. A food product, a feed product, a dietary supplement or a pharmaceutical product, comprising a particle according to claim 12.

16. A food product, a feed product, a dietary supplement or a pharmaceutical product, comprising a particle according to claim 13.
